(19) United States
(12) Patent Application Publication
(10) Pub. No.: US 2021/0054609 A1
(43) Pub. Date: Feb. 25, 2021

(54) Shield and Method of Use Thereof

(71) Applicant: L&B Splash Blocker, LLC, Warminster, PA (US)

(72) Inventors: William Dick, Warminister, PA (US); Linda Dick, Warminister, PA (US)

(73) Assignee: L&B Splash Blocker, LLC, Warminster, PA (US)

(21) Appl. No.: 17/090,010

(22) Filed: Nov. 5, 2020

Related U.S. Application Data
(63) Continuation of application No. 15/559,246, filed on Sep. 18, 2017, now abandoned.
Application No. 15/559,246 is a continuation of application No. 29/537,234, filed on Aug. 24, 2015, now Pat. No. D782635.

(51) Int. Cl. E03D 9/00; A61B 90/00

(52) U.S. Cl. CPC E03D 9/00; F41H 5/08; A61B 90/05

(57) ABSTRACT

A shield apparatus for protecting a person from exposure to a waste material. The shield apparatus includes a body portion and a lip portion. The body portion has a front side and a back side, and is defined by a curved top edge opposite a straight bottom edge, and a straight left edge and a straight right edge joining the curved top edge and the straight bottom edge. The lip portion has a top side and a back side and is defined by a straight front edge opposite a straight back edge, and a straight left edge and a straight right edge joining the straight front edge and the straight back edge. The shield apparatus also includes a handle attached to the front side of the body portion.

### CROSS-REFERENCE TO RELATED APPLICATION

This application claims the priority and benefit of U.S. patent application Ser. No. 15/559,246, titled “Shield and Method of Use Thereof” filed on Sep. 18, 2017, which claims the priority and benefit to International Patent Application PCT/US2015/065150 filed Dec. 11, 2015, which claims the priority and benefit to U.S. Design patent application Ser. No. 29/537,234, titled “Splash Shield,” filed on Aug. 24, 2015, the entireties of which is incorporated herein by reference.

### FIELD OF THE INVENTION

The present invention relates generally to an apparatus for protecting a user from splashes, and particularly to a splash shield for protecting a health care worker from splashes resulting from the disposal of waste material or harmful chemotherapy agents.

### BACKGROUND

In healthcare facilities such as hospitals, nursing homes, etc., health care professionals such as nurses are often required to dispose of waste material by dumping or pouring the waste material into a typical bathroom toilet and then flushing the toilet to empty the toilet bowl. The waste materials often contain substances which are harmful to the health care professional if the health care processional is exposed to the waste material. Exemplary waste materials include human bodily fluids such as blood, urine, fecal matter, vomit, etc., which may include harmful microorganisms, as well as toxic chemotherapy agents. Exposure may result from at least two primary sources. First, the health care professional may be exposed to the waste material while pouring the waste material into the toilet due to splashing. Second, the health care professional may be exposed to the waste material while flushing the toilet, which causes small particles to be emitted into the atmosphere. Exposure may also result from the use of a sprayer which may be either handheld or integral with the toilet for rinsing the toilet bowl or the container used to transport the waste material, such as a bed pan.

Accordingly, it is desirable to provide the health care professional with an apparatus which will prevent exposure to the waste material.

### SUMMARY

According to one embodiment, an apparatus is provided for protecting a health care professional from exposure to a waste material. The apparatus, which is light, portable and able to stand upright, includes a body portion attached to a lip portion. The body portion is defined by a curved top edge opposite a straight bottom edge, and a straight left edge and a straight right edge joining the curved top edge and the straight bottom edge. The body portion has a front side and a back side. The lip portion is defined by a straight front edge opposite a straight back edge, and a straight left edge and a straight right edge joining the straight front edge and the straight back edge. The lip portion has a top side and a bottom side. The front edge of the lip portion is joined to the bottom edge of the body portion. The apparatus further includes a handle attached to the front side of the body portion.

According to another embodiment, a splash shield is provided for protecting a health care professional from exposure to a waste material. The splash shield includes a body portion integral with a lip portion. The angle between the body portion and the lip portion is less than 90 degrees. A handle is further joined to the body portion. The body portion has a front side and a back side, and is defined by a curved top edge opposite a straight bottom edge, and a straight left edge and a straight right edge joining the curved top edge and the straight bottom edge. The lip portion has a top side and a bottom side, and is defined by a straight front edge opposite a straight back edge, and a straight left edge and a straight right edge joining the straight front edge and the straight back edge. The front edge of the lip portion is joined to the bottom edge of the body portion. The right edge of the body portion is joined to the bottom edge of the body portion at a first angle of greater than 90 degrees and the left edge of the body portion is joined to the bottom edge of the body portion at a second angle equal to the second angle. The right edge of the lip portion is joined to the back edge of the lip portion at a third angle of greater than 90 degrees and the left edge of the lip portion is joined to the back edge of the lip portion at a fourth angle equal to the third angle.

According to another embodiment, a method of disposing of a waste material includes transferring the waste material to a bowl of a flush toilet while partially covering the bowl with a splash shield. The splash shield includes a body portion attached to a lip portion. The body portion is defined by a curved top edge opposite a straight bottom edge, and a straight left edge and a straight right edge joining the curved top edge and the straight bottom edge. The body portion has a front side and a back side. The lip portion is defined by a straight front edge opposite a straight back edge, and a straight left edge and a straight right edge joining the straight front edge and the straight back edge. The lip portion has a top side and a bottom side. The front edge of the lip portion is joined to the bottom edge of the body portion. The splash shield further includes a handle attached to the front side of the body portion.

### BRIEF DESCRIPTION OF FIGURES

Additional aspects, features and advantages of the invention, as to its structure, assembly and use, will be understood and become more readily apparent when the invention is considered in light of the following brief description of the figures made in conjunction with the accompanying drawings, wherein:

FIG. 1 is a perspective view of a splash shield, according to an embodiment of the present invention;

FIG. 2 is a front elevation view of the splash shield shown in FIG. 1;

FIG. 3 is a rear elevation view of the splash shield shown in FIG. 1;

FIG. 4 is a side elevation view of the splash shield shown in FIG. 1;

FIG. 5 is another side elevation view of the splash shield shown in FIG. 1 that is opposite the side elevation view shown in FIG. 4;

FIG. 6 is a bottom plan view of the splash shield shown in FIG. 1; and

FIG. 7 is a top plan view of the splash shield shown in FIG. 1.

### DESCRIPTION OF THE INVENTION

The following description of some embodiment(s) is merely exemplary in nature and is in no way intended to limit the invention, its application, or uses.

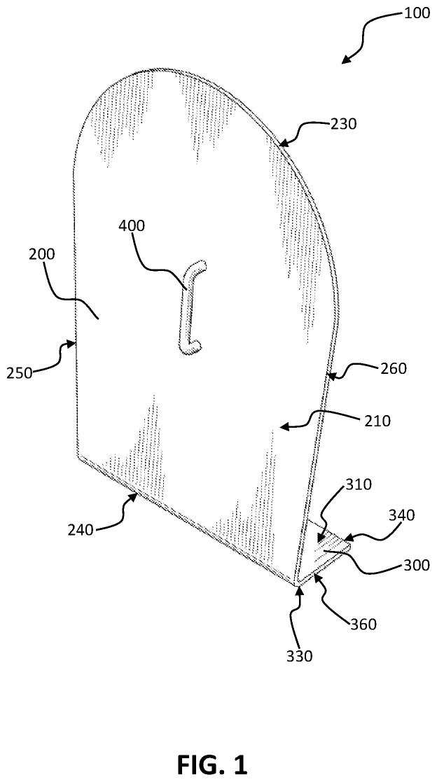

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “left,” “right,” “top” and “bottom” as well as derivatives thereof (e.g., “horizontally,” “downwardly,” “upwardly,” etc.) should be construed to refer to the orientation as described or as shown in FIG. 1. These relative terms are for convenience of description only and do not require that the apparatus be constructed or operated in a particular orientation unless explicitly indicated as such. Terms such as “attached,” “affixed,” “connected,” “coupled,” “interconnected,” “mounted” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments.

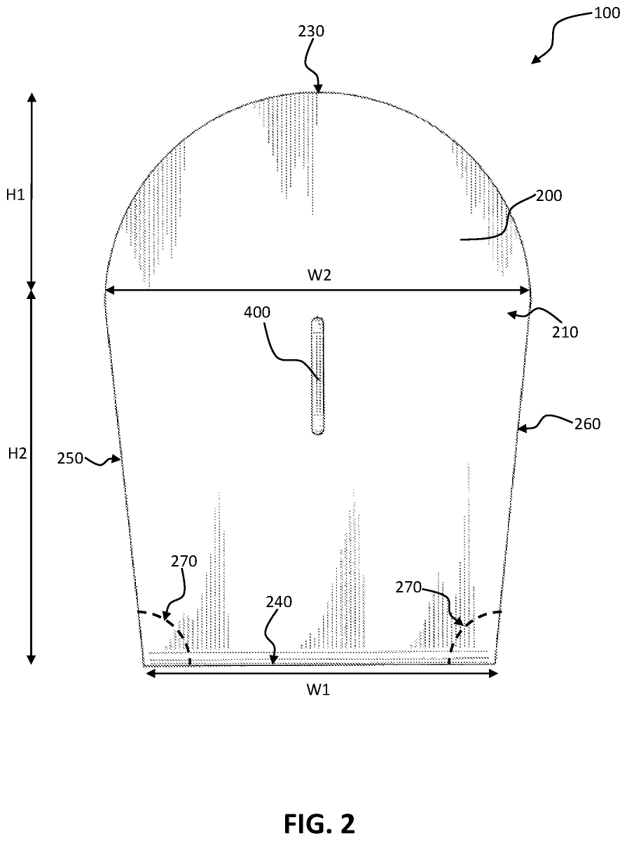

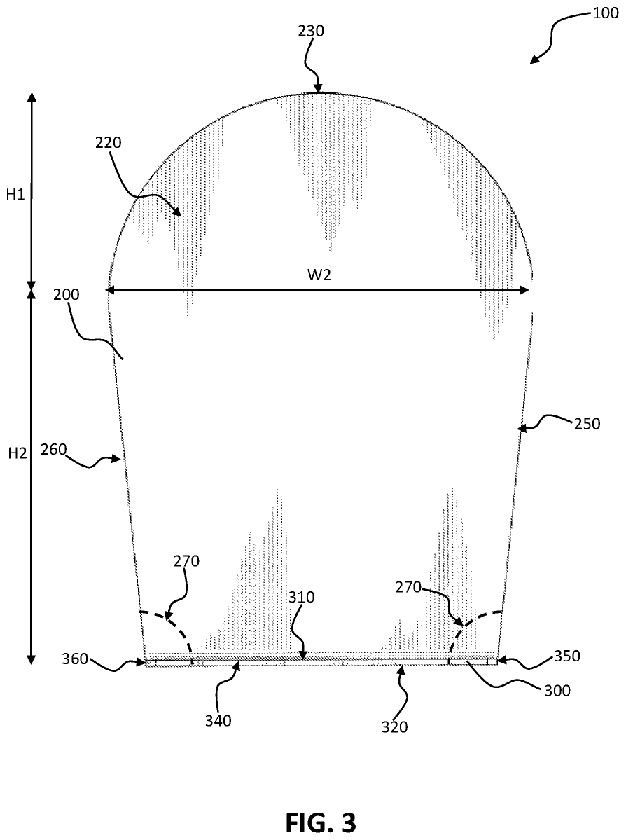

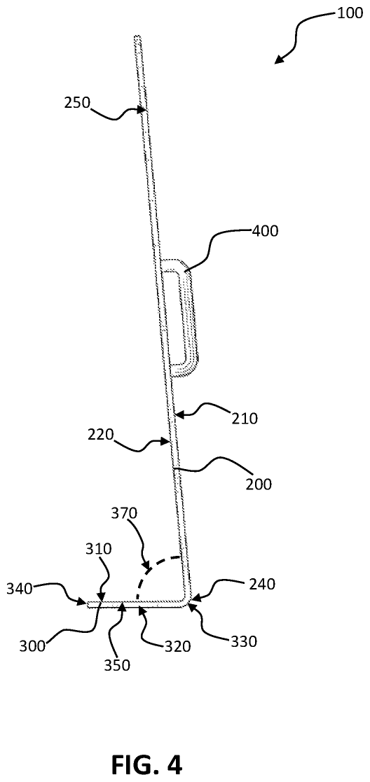

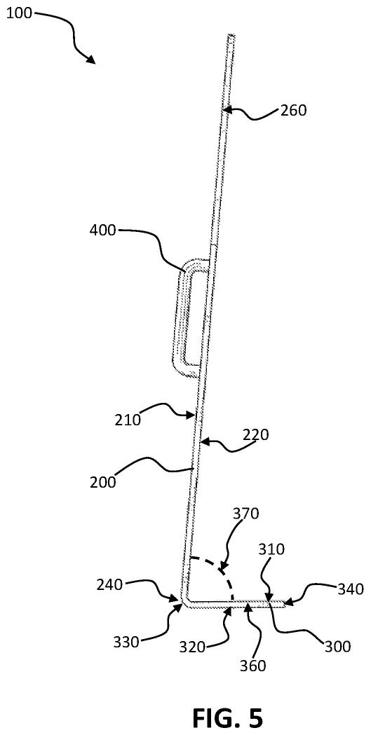

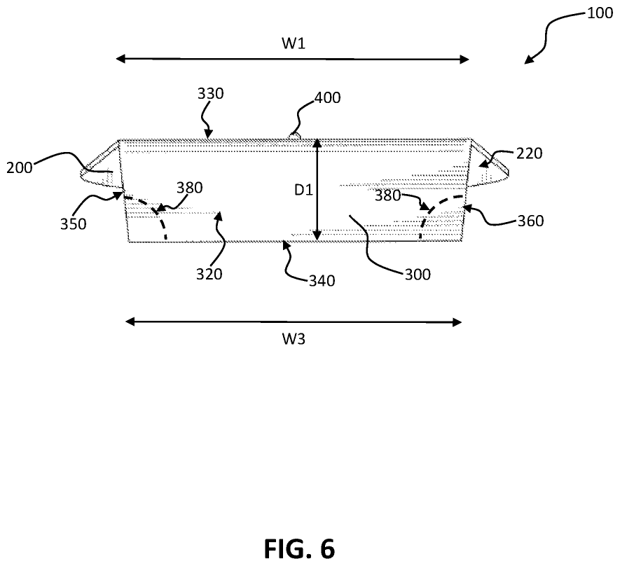

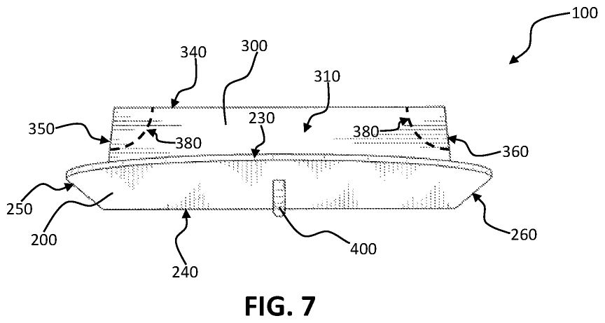

Referring now to the drawings, in which like reference numbers refer to like elements through the various figures that comprise the drawings, FIGS. 1-7 illustrate an exemplary embodiment of a splash shield 100 to protect a health care professional while disposing of waste material in a flush toilet. The splash shield 100 includes a body portion 200 joined to a lip portion 300. The body portion 200 and the lip portion 300 may be integral (as shown in the figures), meaning that they are made of a single piece or a single unitary part without additional pieces. In other embodiments, the body portion 200 and the lip portion 300 may be attached, but not integral. The splash shield 100 further includes a handle 400 attached to, or integral with, the body portion The body portion 200 and the lip portion 300 are further made of a transparent or translucent material, for example a clear acrylic plastic sheet having a thickness of about ⅛ to ¼ of an inch 200 or other thickness so long as the body portion 200 and lip portion 300 maintain structural integrity during use and storage. Other suitable materials include, but are not limited to, polycarbonates, polyesters, etc.

The body portion 200 includes a front side 210 and a back side 220 and is defined by a top edge 230, a bottom edge 240, the left edge 250, and the right edge 260. The top edge 230 is preferably rounded, and the bottom edge 240, a left edge 250, and a right edge 260 are preferably straight. The left edge 250 and the right edge 260 join the bottom edge 240 at an angle 270 which is greater than 90 degrees. As a result, the body portion 200 has a top rounded portion having a height H1 and a bottom portion which is an inverted trapezoid having a bottom width W1, a top width W2 which is greater than the bottom width W1, and a height 112. The total height of the body portion 200 is equal to the height H1 plus the height 112.

The lip portion 300 includes a top side 310 and a bottom side 320 and is defined by a front edge 330, a back edge 340, a left edge 350, and a right edge 360. The front edge 330 is joined to the bottom edge 240 of the body portion 200, and the left edge 350 and the right edge 360 are continuous with the left edge 250 and the right edge 260 of the body portion 200, respectively. The lip portion 300 is joined to the body portion 200 at an angle 370, which is preferably less than 90 degrees. By joining the lip portion 300 to the body portion an angle 370 of less than 90 degrees, the horizontal center of mass (i.e., parallel to the lip portion) is shifted away from the front edge 330 and toward the back edge 340. The center of mass is thus more centered above the lip portion 300, allowing the splash shield 100 to be stood upright on the lip portion 300 with a reduced risk of falling or tipping over. The front edge 330, the back edge 340, the left edge 350, and the right edge 360 are each preferably straight, with the front edge 330 parallel to the back edge 340 and the left edge 350 and the right edge 360 joining the back edge 340 at an angle 380 which is preferably greater than 90 degrees. Accordingly, the lip portion 300 is trapezoidal in shape, with the front edge 330 having a width equal to width W1, which is longer than the width W3 of the back edge 340. The lip portion 300 has depth D1 defined as the distance between the front edge 330 and the back edge 340.

The body portion 200 is sized to fully cover the bowl of typical flush toilet and can even extend beyond the perimeter of the bowl and seat. In an embodiment, the total height of the body portion 200 (i.e., the height H1 plus the height 112) is preferably approximately 21 inches, and the depth of the lip portion 300 is approximately 3 inches. It will be understood, however, that the splash shield 100 is not limited to any particular shape, size or dimension.

The back 220 of the body portion 200 may also be covered in one or more removable layered, peel away sheets. The peel away sheets can extend the duration of use of the splash shield 100 because the external most sheet, which can be worn by physical wear and tear and degraded by exposure to waste materials, can be removed, thereby exposing a new sheet. For example, after using the splash shield 100, a health care professional may remove one of the peel away sheets and dispose of it, exposing a fresh sheet below. The splash shield 100 may then be ready for reuse without requiring any washing or sanitizing.

A health care professional may use the splash shield 100 as described above to protect himself or herself from splashes while disposing of a waste material in a flush toilet. Before being used, the splash shield 100 may be stored sitting upright on the lip portion 300 or hung by the handle 400 or an additional attached hook element (not shown) in the vicinity of the flush toilet. Alternately, the splash shield 100 may include a hole (not shown) which permits the splash shield 100 to be hung from a hook. Because the bottom surface 320 of the lip portion contacts the ground while the top surface 310 faces the flush toilet while disposing of the waste material (as described in greater detail below), the splash shield 100 may be stored while preventing transfer of waste material from the splash shield 100 to the floor or other supporting surface on which the splash shield 100 is placed. The healthcare professional may partially cover the bowl of the toilet with the splash shield 100 while transferring the waste material to the bowl of the toilet such that the body portion 200 of the splash shield is positioned between the body of the health care professional and the toilet with the front side 210 of the body portion 200 facing the health care professional. Because the splash shield 100 is transparent or translucent, the health care professional is able to observe the transfer of the waste material into the bowl of the toilet, which further reduces the likelihood of a splash or spill. The health care professional is able to position and move the splash shield 100 while holding the handle 400 to avoid having to directly contact either the body portion 200 or the lip portion 300. By doing so, any splashes during transfer will contact the back side 220 of the body portion 200 rather than the health care professional. After the waste material is transferred to the bowl of the toilet, the health care professional may flush the toilet while fully covering the toilet bowl with the splash shield 100 to prevent particles of the waste material from being emitted into the atmosphere. The body portion 200 may be at least slightly larger than the profile of the toilet bowl to ensure that the toilet bowl is fully covered while flushing. The lip portion 300 will hook over the toilet bowl while the toilet is flushed to prevent the splash shield 100 from slipping out of position. After use, the splash shield 100 may be cleaned and prepared for reuse by either washing the back 220 of the body portion 200 or removing one of the peel away sheets described above, if present. Accordingly, the splash shield 100 is durable, reusable, and able to withstand wear and tear of repeated use.

The foregoing description of preferred embodiments of the invention should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

What is claimed:

1. An apparatus for protecting a health care professional from exposure to a waste material, the apparatus comprising:
a body portion defined by a curved top edge opposite a straight bottom edge, and a straight left edge and a straight right edge joining the curved top edge and the straight bottom edge, the body portion having a front side and a back side;
a lip portion defined by a straight front edge opposite a straight back edge, and a straight left edge and a straight right edge joining the straight front edge and the straight back edge, the lip portion having a top side and a bottom side, the front edge of the lip portion joined to the bottom edge of the body portion; and
a handle attached to the front side of the body portion.

2. The apparatus of claim 1, wherein the lip portion is joined to the body portion at an angle of less than 90 degrees.

3. The apparatus of claim 1, wherein the right edge of the body portion is joined to the bottom edge of the body portion at a first angle of greater than 90 degrees and the left edge of the body portion is joined to the bottom edge of the body portion at a second angle of greater than 90 degrees.

4. The apparatus of claim 3, wherein the first angle is equal to the second

5. The apparatus of claim 1, wherein the right edge of the lip portion is joined to the back edge of the lip portion at a first angle of greater than 90 degrees and the left edge of the lip portion is joined to the back edge of the lip portion at a second angle of greater than 90 degrees.

6. The apparatus of claim 4, wherein the first angle is equal to the second angle.

7. The apparatus of claim 1, wherein the body portion is integral with the lip portion.

8. The apparatus of claim 1, wherein the body portion and the lip portion are transparent or translucent.

9. The apparatus of claim 1, wherein the body portion and the lip portion are made of a clear acrylic plastic.

10. A splash shield for protecting a health care professional from exposure to a waste material, the splash shield comprising:
a body portion integral with a lip portion, the lip portion forming angle with the body portion of less than 90 degrees; and
a handle joined to the body portion,
wherein the body portion has a front side and a back side, and is defined by a curved top edge opposite a straight bottom edge, and a straight left edge and a straight right edge joining the curved top edge and the straight bottom edge,
wherein the a lip portion defined by a straight front edge opposite a straight back edge, and a straight left edge and a straight right edge joining the straight front edge and the straight back edge, the lip portion having a top side and a bottom side, the front edge of the lip portion joined to the bottom edge of the body portion,
wherein the right edge of the body portion is joined to the bottom edge of the body portion at a first angle of greater than 90 degrees and the left edge of the body portion is joined to the bottom edge of the body portion at a second angle equal to the second angle, and
wherein the right edge of the lip portion is joined to the back edge of the lip portion at a third angle of greater than 90 degrees and the left edge of the lip portion is joined to the back edge of the lip portion at a fourth angle equal to the third angle.

11. The splash shield of claim 10, wherein the body portion and the lip portion are transparent or translucent.

12. The splash shield of claim 11, wherein the body portion and the lip portion are made of a clear acrylic plastic.

13. A method of disposing of a waste material, the method comprising transferring the waste material to a bowl of a flush toilet while partially covering the bowl with a splash shield including:
a body portion defined by a curved top edge opposite a straight bottom edge, and a straight left edge and a straight right edge joining the curved top edge and the straight bottom edge, the body portion having a front side and a back side,
a lip portion defined by a straight front edge opposite a straight back edge, and a straight left edge and a straight right edge joining the straight front edge and the straight back edge, the body portion having a top side and a bottom side, the front edge of the lip portion joined to the bottom edge of the body portion, and
a handle attached to the front side of the body portion.

14. The method of claim 13, further comprising flushing the flush toilet while fully covering the bowl with the splash shield.

15. The method of claim 13, wherein the lip portion of the splash shield is joined to the body portion of the splash shield at an angle of less than 90 degrees.

16. The method of claim 13, wherein the right edge of the body portion of the splash shield is joined to the bottom edge of the body portion of the splash shield at a first angle of greater than 90 degrees and the left edge of the body portion of the splash shield is joined to the bottom edge of the body portion of the splash shield at a second angle of greater than 90 degrees.

17. The method of claim 16, wherein the first angle is equal to the second angle.

18. The method of claim 13, wherein the right edge of the lip portion of the splash shield is joined to the back edge of the lip portion of the splash shield at a first angle of greater than 90 degrees and the left edge of the lip portion of the splash shield is joined to the back edge of the lip portion of the splash shield at a second angle of greater than 90 degrees

19. The method of claim 18, wherein the first angle is equal to the second angle.

20. The method of claim 13, wherein the body portion of the splash shield is integral with the lip portion of the splash shield.
